(19) United States
(12) Patent Application Publication
(10) Pub. No.: US 2021/0346442 A1
(43) Pub. Date: Nov. 11, 2021

(54) WATER-SOLUBLE, FULL-SPECTRUM, CANNABIS COMPLEX AND METHOD OF PRODUCTION

(71) Applicant: MTNM, INC., Albuquerque, NM (US)

(72) Inventors: Eric N. Merryman, Albuquerque, NM (US); Donald R. Bellew, Albuquerque, NM (US)

(21) Appl. No.: 17/103,766

(22) Filed: Nov. 24, 2020

Related U.S. Application Data
(60) Provisional application No. 63/101,233, filed on May 7, 2020.

(51) Int. Cl. A61K 36/00; A61K 47/40; A61K 47/18; A61K 47/10; A61K 47/06; A61K 47/22; A61K 31/05; A61K 31/352

(52) U.S. Cl. CPC A61K 36/00; A61K 47/40; A61K 47/183; A61K 47/10; A61K 47/06; A61K 2236/39; A61K 31/05; A61K 31/352; A61K 2236/31; A61K 2236/333; A61K 47/22

(57) ABSTRACT

Disclosed herein is a process of producing a water-soluble solid including an amino acid-cyclodextrin-cannabinoid complex from extraction of cannabis. The water-soluble solid composition is also disclosed.

### CROSS REFERENCE TO RELATED APPLICATIONS

This application claims the benefit of a related U.S. Provisional Application Ser. No. 63/101,233 filed May 7, 2020, the disclosure of which is incorporated by reference herein in its entirety.

### FIELD

This disclosure relates to cannabinoid complexes and methods of forming them. In particular, to methods of extracting cannabinoids, purification of extracts, and methods of forming water-soluble complexes therewith.

### BACKGROUND

The statements in this section merely provide background information related to the present disclosure and may not constitute prior art.

Cannabis use includes burning and inhalation of dried Cannabis sativa plant material, ingestion of products made from, or containing oils extracted from the Cannabis sativa plant, and the like, and direct ingestion of the oils extracted from the plants.

Cannabis extracts include cannabinoids and may further include various terpenes and flavonoids, all having limited water solubility making the formation of water-soluble cannabinoid formulations problematic.

Emulsions containing cannabis extracts require the addition of emulsifiers and/or surfactants, adding to the complexity. Such emulsions are also prone to separation limiting their use and effectiveness. Emulsions further do not lend themselves to being formulated into dry powders.

There is a need in the art for forming water-soluble cannabinoid complexes which are stable, and with acceptable taste and handling profiles suitable for distribution and consumption.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect of the disclosure, a process comprises the steps of: a) contacting a Cannabis sativa plant comprising a first amount of cannabinoids with a solvent including C₁-C₅ hydrocarbons to produce an extract mixture and separating the Cannabis sativa plant from the extract mixture to form a crude extract including less than or equal to about 1 wt % of triglycerides having C₆-C₃₀ fatty acid residues and/or C₆-C₃₀ fatty acids, based on the total amount of the Cannabis sativa plant present; followed by b) removing the solvent from the crude extract to produce a first extract; c) combining the first extract with an ethanolic solvent to produce a first extract mixture; d) contacting the first extract mixture with a cyclodextrin to produce a second mixture including a cyclodextrin-cannabinoid complex; e) combining the second mixture with an aqueous solution including an amino acid selected from the group consisting of glycine, alanine, serine, or a combination thereof, to produce a final product mixture comprising an amino acid-cyclodextrin-cannabinoid complex; and f) removing the solvent from the final product mixture to produce a final solid product including the amino acid-cyclodextrin-cannabinoid complex, having greater than or equal to about 90 wt % of the cannabinoids present in the first amount of cannabinoids.

In one aspect of the disclosure, a composition comprises the final solid product produced by the process according to one or more aspects of the disclosure.

### DETAILED DESCRIPTION

Cannabis extracts include oils having essentially no appreciable water solubility. One aspect of this disclosure is a process to produce a water-soluble power which includes cannabinoids obtained from the extraction of the Cannabis sativa plant. In an aspect of the disclosure, the water-soluble power also includes various flavonoids, terpenes, and other materials originally present in the Cannabis sativa plant, increasing the benefits inherent in consumption of these materials. Such extracts are referred to in the art as being “full-spectrum”. In aspects of the disclosure, the Cannabis sativa plant is extracted, the extract is purified, and then the components of the extract are complexed with cyclodextrin to form a water-soluble matrix. In aspects of the disclosure, this water-soluble matrix if further encapsulated with an amino acid, e.g., glycine, which both stabilizes the complex and imparts a sweetness to these otherwise bitter tasting oils.

### Definitions

For purposes herein, and the claims thereto, the new numbering scheme for the Periodic Table Groups is used as described in Chemical and Engineering News, 63(5), pg. 27 (1985). Therefore, a “group 4 metal” is an element from group 4 of the Periodic Table, e.g. Hf, Ti, or Zr.

As used herein, and unless otherwise specified, the term “Cₙ” means hydrocarbon(s) having n carbon atom(s) per molecule, where n is a positive integer. Likewise, a “Cₘ-C_{y}” group or compound refers to a group or compound comprising carbon atoms at a total number thereof in the range from m to y. Thus, a C₁-C₄ alkyl group refers to an alkyl group that includes carbon atoms at a total number thereof in the range of 1 to 4, e.g., 1, 2, 3 and 4.

“Moiety” refers to one or more covalently bonded atoms which form a part of a molecule. The terms “group,” “radical,” “moiety”, and “substituent” may be used interchangeably.

The terms “hydrocarbyl radical,” “hydrocarbyl group,” or “hydrocarbyl” may be used interchangeably and are defined to mean a group consisting of hydrogen and carbon atoms only. Preferred hydrocarbyls are C₁-C₂₀ radicals that may be linear, branched, or cyclic, and when cyclic, aromatic or non-aromatic. Examples of such radicals include, but are not limited to, alkyl groups such as methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and the like, aryl groups, such as phenyl, benzyl naphthyl, and the like.

For purposes herein, a heteroatom is any non-carbon atom, selected from groups 13 through 17 of the periodic table of the elements. In one or more aspects, heteroatoms are non-metallic atoms selected from B, N, O, Si, P, S, As Se, Te and the halogens F, Cl, Br, I, and At.

Unless otherwise indicated, the term “substituted” means that at least one hydrogen atom has been replaced with at least one non-hydrogen atom or a functional group.

For purposes herein, a functional group includes one or more of a hydrocarbyl group, a heteroatom, or a heteroatom containing group, such as halogen (such as Br, Cl, F or I) or at least one functional group such as —NR*₂, —NR*—CO—R*, —OR*,*—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —(CH₂)q-SiR*₃, or a combination thereof, where q is 1 to 10 and each R* is independently hydrogen, a hydrocarbyl or halocarbyl radical, and two or more R* may join together to form a substituted or unsubstituted completely saturated, partially unsaturated, or aromatic cyclic or polycyclic ring structure, or where at least one heteroatom has been inserted within a hydrocarbyl ring.

A heterocyclic ring, also referred to herein as a heterocyclic radical, is a ring having a heteroatom in the ring structure as opposed to a heteroatom substituted ring where a hydrogen on a ring atom is replaced with a heteroatom. For example, tetrahydrofuran is a heterocyclic ring and 4-N,N-dimethylamino-phenyl is a heteroatom substituted ring. A substituted heterocyclic ring is a heterocyclic ring where a hydrogen of one of the ring atoms is substituted, e.g., replaced with a hydrocarbyl, or a heteroatom containing group.

A “compound” refers to a substance formed by the chemical bonding of a plurality chemical elements. A “derivative” refers to a compound in which one or more of the atoms or functional groups of a precursor compound have been replaced by another atom or functional group, generally by means of a chemical reaction having one or more steps.

For any particular compound disclosed herein, any general or specific structure presented also encompasses all conformational isomers, regio-isomers, and stereoisomers that may arise from a particular set of substituents, unless stated otherwise. Similarly, unless stated otherwise, the general or specific structure also encompasses all enantiomers, diastereomers, and other optical isomers whether in enantiomeric or racemic forms, as well as mixtures of stereoisomers, as would be recognized by a skilled artisan.

As used herein, the term “aromatic” also refers to pseudoaromatic heterocycles which are heterocyclic substituents that have similar properties and structures (nearly planar) to aromatic heterocyclic ligands, but are not by definition aromatic; likewise the term aromatic also refers to substituted aromatics.

As used herein, a moiety which is chemically identical to another moiety is defined as being identical in overall composition exclusive of isotopic abundance and/or distribution, and/or exclusive of stereochemical arrangement such as optical isomers, confirmational isomers, spatial isomers, and/or the like.

As used herein a mixture refers to a combination of the various components which may be heterogeneous or homogeneous. In contrast a solution refers to a homogenous combination wherein the solute is entirely dissolved in the solvent forming a clear solution. Importantly, ink is a clear liquid, milk is a colorless liquid, and water is a clear-colorless liquid.

Homogenization and/or mixing under homogenizing conditions refers to one or more processes wherein larger particles of a mixture component are physically reduced in size to smaller particles of uniform size which distribute evenly in the other components present resulting in a uniform mixture. Homogenization may include a French pressure press in which a mixture is forced at relatively high pressure of several thousand psi through small holes, the use of extruders, various types of mechanical grinding mills, the use of colloid mills in which a rotor turns at high speeds e.g., 2000-18,000 rpm applying a relatively high level of hydraulic shear and other stresses on the fluid thereby disrupting and breaking down the structure of the materials present in the mixture. Homogenization may also include sonication in which sound energy is applied to the mixture at ultrasonic frequencies e.g., greater than about 20 kHz, typically involving the use of an ultrasonic probe in the mixture or placing the mixture in an ultrasonic bath.

For purposes herein, absolute ethanol refers to ethanol having less water than is obtained via azeotropic distillation, e.g., less than about 4.4 wt % water, and may include ethanol having less than or equal to about 1 wt % water.

As used herein, the Cannabis sativa plant, also referred to herein simply as the “plant” refers to the plants in the Cannabaceae family and the Cannabis genus. Specific examples of species include Cannabis sativa, Cannabis indica, Cannabis ruderalis, and those generally referred to as Cannabis C. sativa or simply C. sativa, and the like.

As used herein, cannabinoids include both optically pure and racemic pairs of compounds which may be isolated from one or more of the cannabis plants including chemotypes I, II, III, and the like. Suitable cannabinoids for purposes herein may be isolated from the cannabis plant e.g., the Cannabis sativa plant, and/or may be synthetically produced and/or modified, and/or biosynthesized.

### Formation of Water-Soluble Cannabinoid Complexes

In one aspect, the process to produce a water-soluble cannabinoid complex comprises an extraction step a) followed by a solvent removal step b) to form an extract, followed by a complexing step in which the extract is dissolved in a solvent step c) and contacted with a cyclodextrin in a complexing step d) to form a cyclodextrin-cannabinoid complex, followed by an encapsulation step e) in which the complex is encapsulated with one or more amino acids, followed by a solid producing step f) in which solvent from the process is removed and the final water-soluble complex is formed.

### Extraction Step a)

In some aspects, the extraction step includes contacting a Cannabis sativa plant comprising a first amount of cannabinoids with a solvent including C₁-C₅ hydrocarbons. In one aspect the cannabis plants include Cannabis sativa, cannabis indica, Cannabis ruderalis, cannabis C. sativa and/or the like, and may include Cannabis sativa plants of chemotypes I, II, III, and the like. In some aspects, the plant material utilized in the extraction includes the leaves, buds, stalks, and/or the like. In other aspects, certain portions of the Cannabis sativa plant are utilized, e.g., buds, flowering tissues, leaves, and/or the like. In some aspects, the plant or plants to be used in the extraction are refrigerated directly after being harvested and maintained under refrigeration until the extraction step. In one aspect, the Cannabis sativa plant, or portions thereof, has been maintained at a temperature of less than or equal to about 10° C., or less than or equal to about 5° C., or less than or equal to about 0° C., or less than or equal to about −10° C., or less than or equal to about −20° C., e.g., frozen, from a time of less than 24 hrs after harvest to the time of the extraction. In other aspects, the plants are refrigerated and/or frozen within about 12 hrs, or within 4 hours, or within 1 hr of harvest. In other aspects, the plants are essentially immediately refrigerated after harvest. In one aspect of the disclosure, the harvested plant material is flash frozen with dry ice, CO₂ freezing, liquid nitrogen, and/or the like directly after being harvested. In addition, the harvested material is preferably kept out of direct sunlight e.g., kept in the dark, until extracted.

In one aspect, the extraction includes contacting the plant material with a solvent, preferably a hydrocarbon solvent, preferably a C₁-C₅ hydrocarbon, to produce an extract mixture. In one aspect the plant material is contacted with propane, butane, or a combination thereof. Applicant has discovered that propane results in a higher terpene content in the final product, while butane results in a higher cannabinoid content in the final product. In some aspects, the extraction includes agitation of the mixture. In other aspects, the mixture is not agitated during the extraction process.

In one aspect, the extraction includes contacting the plant material with the solvent at a temperature of less than or equal to about 40° C., or less than or equal to about 35° C., or less than or equal to about 30° C., or less than or equal to about 25° C., or less than or equal to about 20° C., or less than or equal to about 15° C., or less than or equal to about 10° C., or less than or equal to about 5° C., with a temperature of less than or equal to about 0° C. being preferred. In some aspects, the extraction includes contacting the plant material with the solvent at the extraction temperature for less than or equal to about 30 min, or less than or equal to about 15 min, or less than or equal to about 10 min, or less than or equal to about 7 min, or less than or equal to about 5 min, or less than or equal to about 4 min, followed by separating the plant material from the solvent to produce the crude extract.

The extraction step may take place above atmospheric pressure, preferably at a pressure suitable to prevent significant loss of the solvents. Likewise, the extraction vessel may be cooled during the extraction process to maintain a suitable temperature.

In some aspects, the plant material is contacted with the solvent including C₁-C₅ hydrocarbons to produce the extract mixture under conditions sufficient to produce an extract having less than or equal to about 1 wt %, or 0.5 wt %, or 0.1 wt % of triglycerides having C₆-C₃₀ fatty acid residues and/or C₆-C₃₀ fatty acids, based on the total amount of the plant material present in the extraction. For example, if 100 g of plant material is extracted, the total amount of triglycerides having C₆-C₃₀ fatty acid residues and/or C₆-C₃₀ fatty acids present in the crude extract produced would be less than 1 g, or 0.5 g, or 0.1 g, respectively.

In one aspect of the disclosure, the crude extract is produced by repeating step the contacting of the plant material with the solvent followed by separating of the solvent in step (a) a plurality of times using the same plant material and fresh solvent each time the material is extracted to produce a partial crude extract, followed by combining the plurality of partial crude extracts to form the crude extract.

In one aspect of the disclosure, an extraction column may be utilized in which the plant material is contacted with the solvent. In some aspects, a counter-current extraction process may be utilized in which the plant material is directed through an extraction device counter currently to the extraction solvent being directed through the extraction device in a continuous or semi-continuous mode of operation. The crude extract then being separated from the extracted plant material, e.g., via filtration, to produce the crude extract.

In one aspect, the process further includes capturing at least a portion of the solvent removed during one or more steps, optionally purifying the solvent for reuse, and then directing at least a portion of the recovered solvent back into the process.

### Solvent Removal Step b)

After forming the crude extract during the extraction step, the next step is to remove the solvent from the crude extract. The end result of this solvent removal step is a first extract, typically in the form of an oil. In one aspect of the disclosure, solvent removal includes distillation of the of the crude extract to remove the hydrocarbon solvent, typically at a temperature of less than or equal to about 40° C., or 35° C., or 30° C., or 27° C., at atmospheric pressure. The hydrocarbon solvent may then be recovered and reused. In some aspects of the disclosure, the solvent removal step further includes one or more processes to further remove solvent from the resultant oil extract. In one aspect, after the bulk of the hydrocarbon solvent is removed the resultant oil is maintained at a temperature for less than or equal to about 20° C., or 15° C., or 10° C., while being purged with a gas. In some aspects, the oil is purged at atmospheric pressure with nitrogen, compressed air, argon, and/or a combination thereof. In some aspects, solvent present in the oil extract is further removed at elevated temperature at reduced pressure. In one aspect, either with or without purging, the oil is maintained at a temperature from about 37° C. to about 65° C., or from about 40° C. to about 52° C., at a pressure of less than or equal to about 100 torr, or 80 torr, or 60 torr, or 40 torr for a period of time from about 0.1 hour to 100 hours, or from about 1 hour to 90 hours, or from about 24 hrs to 72 hrs (e.g., from about 1 to 3 days).

Removal of the solvent from the crude extract results in the formation of the first extract. In one aspect of the disclosure, the first extract includes less than or equal to about 10 ppm hydrocarbon solvent, or less than or equal to about 1 ppm hydrocarbon solvent, or less than or equal to about 0.10 ppm hydrocarbon solvent, as determined via gas chromatography, based on the total amount of the extract present.

### Complex Formation

Next a complex is formed between the extract and a complexing agent. In one aspect, the complexing step includes dissolving of the extract in a solvent step c) and contacted of the dissolved extract with a cyclodextrin in complexing step d) to form a cyclodextrin-cannabinoid complex.

In one aspect of the disclosure, the first extract is combined with a suitable solvent to produce a first extract mixture. In one aspect of the disclosure, the first extract is dissolved in an ethanolic solvent with mixing, which in another aspect of the disclosure, the solvent is, i.e., consists essentially of, or consists of absolute ethanol. In one aspect of the disclosure, the first extract is dissolved in an amount of absolute ethanol sufficient to form a clear homogeneous solution, e.g., a clear solution when determined at 25° C.

Importantly, applicant has discovered that a minimum amount of solvent is preferably utilized in this step to increase the formation of the complex in subsequent steps, and to reduce the amount of solvent which must be subsequently removed and optionally, recovered.

In one aspect of the disclosure, the first extract mixture is produced by combining the first extract with the solvent and filtering the mixture to produce the first extract mixture, which is preferably a clear solution.

### Complexing Step d)

In one aspect of the disclosure, the first extract mixture is contacted with a complexing agent, which in some aspects of the disclosure is a cyclodextrin, to produce a second mixture including a complexing agent—cannabinoid complex, which may be a cyclodextrin-cannabinoid complex. In some aspects, step d) includes combining the first extract mixture with an amount of cyclodextrin dissolved in an ethanolic solvent, which may be the cyclodextrin dissolved in absolute ethanol. In some aspects, the cyclodextrin is dissolved in an amount of absolute ethanol necessary to form a clear solution at 25° C.

In one aspect of the disclosure, the complexing agent includes a cyclodextrin which includes one or more cyclodextrins. In some aspects of the disclosure, the one or more cyclodextrins is (are) further substituted with one or more functional groups selected from the group consisting of C₁-C₂₀ saturated hydrocarbon radicals, C₃-C₂₀ unsaturated hydrocarbon radicals, C₆-C₂₀ aromatic hydrocarbon radicals, C₅-C₂₀ heterocyclic hydrocarbon radicals, Br, Cl, F, I, —NR*₂, —NR*—CO—R*, —OR*,*—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, where q is 1 to 10 and each R* is independently hydrogen, a C₁-C₂₀ radical, a substituted C₁-C₂₀ radical, and/or two or more R* may join together to form a substituted or unsubstituted completely saturated, partially unsaturated, aromatic, cyclic, or polycyclic ring structure.

In one aspect of the disclosure, the cyclodextrin is selected from the group consisting of randomly methylated beta-cyclodextrin, randomly methylated gamma-cyclodextrin, 2-hydroxypropyl-beta-cyclodextrin, 2,3-dihydroxypropyl-beta-cyclodextrin, 2-hydroxypropyl-gamma-cyclodextrin, 2,3-dihydroxypropyl-gamma-cyclodextrin, sulfobutyl ether-beta-cyclodextrin, sulfobutyl ether-gamma-cyclodextrin, polymeric cyclodextrin, or a combination thereof. In another aspect of the disclosure, the cyclodextrin includes, or is hydroxypropyl-beta-cyclodextrin.

The extract mixture is combined with the complexing agent, e.g., the cyclodextrin, under homogenizing conditions at a temperature, and for a period of time sufficient to form a second mixture. In one aspect of the disclosure, the first extract mixture is contacted with the cyclodextrin under homogenizing conditions at a temperature of less than or equal to about 40° C., or less than or equal to about 35° C., or less than or equal to about 30° C., or less than or equal to about 25° C., or less than or equal to about 15° C. for about 0.1 to 100 hours, or from about 0.5 to 5 hours, or from about 1 to 3 hours. In one aspect of the disclosure, the extract mixture is combined with the complexing agent under homogenizing conditions which include sonication and/or utilizing a high speed impeller-stator type homogenizer at greater than or equal to about 10,000 RPM, or 13,000 RPM, or 15,000 RPM, or 18,000 RPM, according to practices of homogenization known in the art.

In one aspect of the disclosure, step d) includes combining the first extract mixture with cyclodextrin dissolved in absolute ethanol under homogenizing conditions at a temperature below about 30° C. to form the second mixture including a cyclodextrin-cannabinoid complex, wherein the second mixture is a clear solution at 25° C. In some aspects of the disclosure, after the homogenization, the mixture may be filtered to produce the second mixture including a cyclodextrin-cannabinoid complex, which is preferably a clear solution at 25° C.

In one aspect of the disclosure, the amount of the complexing agent utilized to form the complex is determined, and a slight excess, if any of the complexing agent is utilized to form the second mixture including the cyclodextrin-cannabinoid complex. In one aspect of the disclosure, an average molecular weight of the first extract is determined. The molar amount of material present in the first extract is then calculated. The amount of the complexing agent required is then determined based on a mole/mole ratio of extract to complexing agent. This amount of complexing agent is then combined with first extract as discussed herein to form the complexing agent-cannabinoid complex. Applicant has discovered that too little of the complexing agent results in an oil residue after the complexing step, while an excess amount of the complexing agent is wasteful and dilutes the active material in the final product.

For example, analysis of a first extract may show a particular combination of components which may include various cannabinoids, terpenes, flavonoids, and/or the like. The weight percentage of each of these components is converted into a mol % of each component, and an average molecular weight of the extract is then determined. This average molecular weight of the first extract is then multiplied by the mass of the first extract which is then multiplied by the molecular weight of the complexing agent and divided by the mole:mole ratio of component to complex required by the complexing agent to determine the amount of complexing agent to use. In one aspect of the disclosure, the mole:mole ratio of first extract to cyclodextrin is from about 1:1 to 1:2, or from about 1:1.05 to 1:1.5, or from about 1:1.1 to 1:1.3 calculated moles present in extract to moles of cyclodextrin.

In one example, an average molecular weight of a first extract was determined to be about 280 g/mol. 50 g of this extract thus represented about 0.27 mols of the extract. A hydroxypropyl beta cyclodextrin having a molecular weight of about 1396 g/mol was combined in a 1:1.05 mol ratio with the extract resulting in the need for about 373 g of the hydroxypropyl beta cyclodextrin.

### Encapsulation Step e)

The second mixture which includes the cyclodextrin-cannabinoid complex is then combined with an aqueous solution including an amino acid, which in one aspect of the disclosure is an amino acid selected from the group consisting of glycine, alanine, serine, or a combination thereof, to produce a final product mixture comprising an amino acid-cyclodextrin-cannabinoid complex. In one aspect of the disclosure, the complexing agent-cannabinoid complex, which in one aspect is a cyclodextrin-cannabinoid complex is encapsulated, or at least partially encapsulated by the amino acid forming, for example the amino acid-cyclodextrin-cannabinoid complex. In one aspect the complex includes or is a glycine-hydroxypropyl-beta-cyclodextrin-cannabinoid complex.

In one aspect of the disclosure, the aqueous solution including the amino acid includes less than about 10 wt % of the amino acid, or less than about 7 wt % of the amino acid, or less than about 5 wt % of the amino acid, or less than about 1 wt % of the amino acid, which is preferably glycine, alanine, serine, or a combination thereof in water. In one aspect of the disclosure, the aqueous solution is from about 1 to 10 wt % glycine in water.

The amino acid solution is added to the cyclodextrin-cannabinoid complex under homogenization conditions which may be similar to those utilized in forming the cyclodextrin-cannabinoid complex. In one aspect of the disclosure, the aqueous amino acid solution including less than or equal to about 10 wt % glycine is combined with the second mixture including the cyclodextrin-cannabinoid complex under homogenizing conditions at a temperature, and for a period of time sufficient to form a final product mixture. In an aspect of the disclosure, the final product mixture is an emulsion.

In one aspect of the disclosure, the second mixture is contacted with the amino acid solution under homogenizing conditions at a temperature of less than or equal to about 40° C., or less than or equal to about 35° C., or less than or equal to about 30° C., or less than or equal to about 25° C., or less than or equal to about 15° C. for about 0.1 to 100 hours, or from about 0.5 to 5 hours, or from about 1 to 3 hours. In one aspect of the disclosure, the second mixture is combined with the aqueous amino acid solution under homogenizing conditions which include sonication and/or utilizing a high speed impeller-stator type homogenizer at greater than or equal to about 10,000 RPM, or 13,000 RPM, or 15,000 RPM, or 18,000 RPM, according to practices of homogenization known in the art.

In some aspects of the disclosure the amount of amino acid combined with the second solution is based on an estimated cannabinoid concentration present in the first extract. In one aspect, the amount of amino acid utilized is greater than or equal to about 1 g of amino acid per 50 g of cannabinoid present in the first extract, or greater than or equal to about 1 g of amino acid per 75 g of cannabinoid present in the first extract, or greater than or equal to about 1 g of amino acid per 100 g of cannabinoid present in the first extract, or greater than or equal to about 1 g of amino acid per 150 g of cannabinoid present in the first extract.

In one or more aspects of the disclosure, the final product mixture is an emulsion which is stable for about 1 hour at 25° C. In some aspects, the mixture including the amino acid-cyclodextrin-cannabinoid complex may be filtered to produce the final product mixture as a clear solution. In alternative aspects, the mixture including the amino acid-cyclodextrin-cannabinoid complex is utilized as an emulsion without any subsequent filtering or other processing.

### Solid Producing Step f)

In one aspect of the disclosure, the solvent present in the final product mixture including the amino acid-cyclodextrin-cannabinoid complex is removed to form a water-soluble solid including the amino acid-cyclodextrin-cannabinoid complex. This solid material may then be masticated, e.g., grinding, milling, air jet milling, and/or the like, to form a free flowing water-soluble powder including the amino acid-cyclodextrin-cannabinoid complex. In one or more aspects, the solid product including the amino acid-cyclodextrin-cannabinoid complex (also referred to herein as the “final solid product”) is formed by evaporation of the solvent under reduced pressure, by lyophilization, by freeze drying, by spray drying, filter drying, prilling, fluidized bed drying, and/or the like. In one aspect of the disclosure, the solvent present in the final product mixture is removed at temperature of less than or equal to about 40° C., or less than or equal to about 30° C., or less than or equal to about 20° C., or less than or equal to about 10° C., or less than or equal to about 5° C. at a pressure below atmospheric pressure, over a period of time of about 0.1 to 100 hours, or from about 1 to 50 hours, or from about 5 to 24 hours.

In some aspects of the disclosure, the resultant final solid product is milled at a temperature of less than or equal to about 40° C., or less than or equal to about 30° C., or less than or equal to about 20° C., or less than or equal to about 10° C., or less than or equal to about 5° C. to produce a free-flowing powder.

In one aspect of the disclosure, a 10 wt % mixture of the final solid product in water forms a clear solution at 25° C. In other aspects of the disclosure, a 15 wt % mixture of the final solid product in water, or a 20 wt % mixture of the final solid product in water, or a 25 wt % mixture of the final solid product in water, or a 30 wt % mixture of the final solid product in water forms a clear solution at 25° C.

### Water-Soluble Solid Product

The Cannabis sativa plant (e.g., the plant material) utilized to produce the final solid product according to aspects of the disclosure naturally includes a first amount of cannabinoids. Likewise, the plant material naturally includes a first amount of terpenes and/or flavonoids. In one aspect of the disclosure, the final solid product of the process includes greater than or equal to about 90 wt % of this first amount of cannabinoids. In another aspect of the disclosure, the final solid product of the process includes greater than or equal to about 90 wt % of this first amount of cannabinoids and/or the first amount of terpenes and/or flavonoids. In some aspects of the disclosure, the final solid product of the process includes greater than or equal to about 95 wt %, or 98 wt % or 99 wt % of this first amount of cannabinoids and/or the first amount of terpenes and/or flavonoids.

In one aspect of the disclosure, the final solid product includes one or more of yangonin, epigallocatechin gallate, dodeca-2E,4E,8Z,10Z-tetraenoic acid isobutylamide, dodeca-2E,4E-dienoic acid isobutylamide, beta-myrcene, alpha-pinene, cis-ocimene, beta-curcumene, terpineol, camphene, trans-20 ocimene, alpha-terpinene, piperitone, eucalyptol, linalool, citronellol, beta-pinene, borneol, citronellal, geraniol, d/l-fenchone, d-limonene, 3-carene, geranyl acetate, cuminaldehyde, alpha-phellandrene, alpha-thujone, d/l-menthol, linalyl acetate, isopulegol, carvone, cavacrol, gamma-terpinene, nerol, menthofuran, sabinene hydrate, sabinene, thymol, camphor, pulegone, bornyl acetate, alpha-terpinol, meta-cymene, cannabispiran, isocannabispiran, beta-farnesene, isoborneol, cis-citral, beta-caryophylene, beta-caryophylene oxide, ledene, alpha-humlene, beta-cedrene, alpha-bisabolol, valencene, cedral, farnesol, cuparene, gauiol, thujopene, phloroglucinol, cannabistillbene, 2-carene, quercetin, cannflavin C, isocannabispiran, luteolin, vitexin, cannflavin A, cannflavin B, cytisoide, apigenin, apigenin-glucoside, isovitexin, dihydo-resveratrol, kaempferol, terpinolene, and vincenin-2.

In one aspect of the disclosure, the final solid product includes one or more of d-limonene, beta-pinene, alpha-pinene, beta-myrcene, linalool, beta-caryophylene, alpha-humlene, citronellol, terpinolene, borneol, carvone, 2-piperidone, nerolidol, carene, ocimene, cymene, eucalyptol, and pulegone. In one or more aspects, the final solid product includes greater than or equal to about 90 wt % of first amount of d-limonene, beta-pinene, alpha-pinene, beta-myrcene, linalool, beta-caryophylene, alpha-humlene, citronellol, terpinolene, borneol, carvone, 2-piperidone, nerolidol, carene, ocimene, cymene, eucalyptol, and/or pulegone originally present in the plant material extracted.

In one aspect of the disclosure, the final product includes one or more cannabinoids, which may include one or more of the cyclized and/or uncyclized, substituted and/or unsubstituted forms of:

i) cannabigerol, according to the general formula:

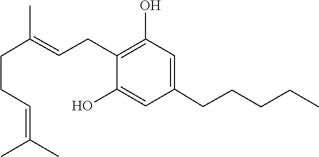

ii) cannabichromene, according to the general formula:

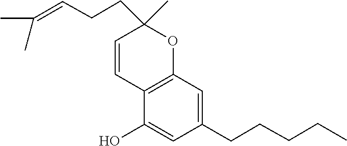

iii) cannabidiol, according to the general formula:

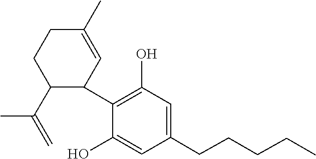

iv) tetrahydrocannabinol and/or cannabinol, according to the general formula:

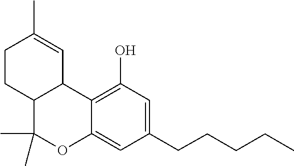

v) cannabielsoin, according to the general formula:

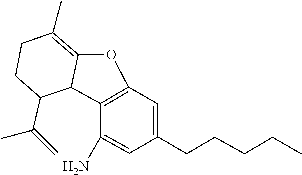

vi) iso-tetrahydrocannabinol, according to the general formula:

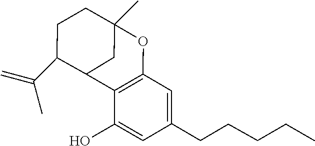

vii) cannabicyclol, according to the general formula:

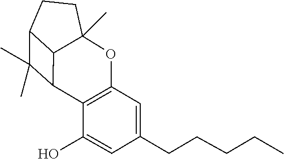

viii) cannabicitran, according to the general formula:

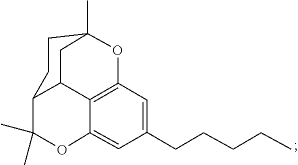

ix) tetrahydrocannabivarin (THCV), according to the general formula:

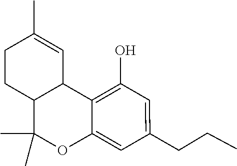

wherein any one or more of the various hydrogen atoms may be substituted with a functional group, and/or including the free acids, salts, tosylates, mesylates, esters, amides, ethers, sulfates, and/or other derivatives thereof.

In one aspect of the disclosure, the final solid product includes one or more tetrahydrocannabinols (THC) in general, and (−)-trans-Δ⁹-tetrahydrocannabinol in particular, cannabidol (CBD), tetrahydrocannabinolic acid (THCA), cannabidiolic acid (CBDA), cannabinol (CBN), cannabigerol (CBG), cannabichromene (CBC) cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), cannabicitran (CBT), cannabigerolic acid, cannabigerolic acid monomethylether, cannabigerol monomethylether, cannabigerovarinic acid, cannabichromenic acid, cannabichromevarinic acid, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-isotetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxycannabitriolvarin, dehydrocannabifuran, cannabifuran, cannabichromanon, cannabicitran, 10-oxo-delta-6a-tetrahydrocannabinol, delta-9-cistetrahydrocannabinol, 3, 4, 5, 6-tetrahydro-7-hydroxy-alₚha-alₚha-2-trimethyl-9-npropyl-2, 6-methano-2H-1-benzoxocin-5-methanol-cannabiripsol, trihydroxy-delta-9-tetrahydrocannabinol, cannabinol, and/or derivatives thereof.

In one or more aspects the phenolic hydrogen, when present, is replaced by a C₁-C₄₀ hydrocarbyl, preferably a C₃-C₄₀ carbohydrate, saccharide or polysaccharide, optionally comprising one or more functional groups, e.g., an aminosaccharide, a decasaccharide, a disaccharide, a glucosaccharide, a heptasaccharide, a heterosaccharide, a hexasaccharide, an isomaltosaccharide, a monosaccharide, an oligosaccharide, a pentasaccharide, a phosphosaccharide, a polysaccharide, a tetrasaccharide, a trisaccharide, a triose, tetrose, a pentose, a hexose, a heptose, a glycoside, and/or the like.

In one or more aspects the cannabinoid comprises both substituted and unsubstituted forms of cannabidiol (CBD) according to the general formula:

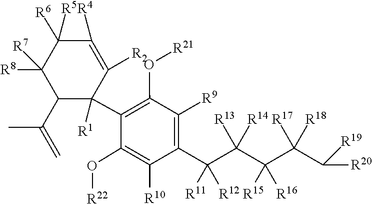

wherein one or more of R¹, R², R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ and R²², are independently selected from hydrogen or one or more monovalent radicals including hydrocarbyl radicals such as methyl, ethyl, ethenyl, and all isomers (including cyclics such as cyclohexyl) of propyl, butyl, pentyl, hexyl, heptyl, octyl, nonyl, decyl, undecyl, dodecyl, propenyl, butenyl, and from halocarbyls and all isomers of halocarbyls including perfluoropropyl, perfluorobutyl, perfluoroethyl, perfluoromethyl, and from substituted hydrocarbyl radicals and all isomers of substituted hydrocarbyl radicals including trimethylsilylpropyl, trimethylsilylmethyl, trimethylsilylethyl, and from phenyl, and all isomers of hydrocarbyl substituted phenyl including methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, dipropylmethylphenyl, and the like; from all isomers of halo substituted phenyl (where halo is, independently, fluoro, chloro, bromo and iodo) including halophenyl, dihalophenyl, trihalophenyl, tetrahalophenyl, and pentahalophenyl; and from all isomers of halo substituted hydrocarbyl substituted phenyl (where halo is, independently, fluoro, chloro, bromo and iodo) including halomethylphenyl, dihalomethylphenyl, (trifluoromethyl)phenyl, bis(triflouromethyl)phenyl; and from all isomers of benzyl, and all isomers of hydrocarbyl substituted benzyl including methylbenzyl, dimethylbenzyl.

In one or more aspects R²¹ and/or R²², comprise a C₃-C₄₀ carbohydrate, saccharide or polysaccharide, optionally comprising one or more functional groups, e,g, an aminosaccharide, a decasaccharide, a disaccharide, a glucosaccharide, a heptasaccharide, a heterosaccharide, a hexasaccharide, an isomaltosaccharide, a monosaccharide, an oligosaccharide, a pentasaccharide, a phosphosaccharide, a polysaccharide, a tetrasaccharide, a trisaccharide, a triose, tetrose, a pentose, a hexose, a heptose, a glycoside, and/or the like.

In one or more aspects, one or more of R¹, R², R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ and R²², is substituted with one or more functional groups selected from Br, Cl, F, I, —NR*2, —NR*—CO—R*,—OR*,*—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, wherein q is 1 to 10 and each R* is independently hydrogen, a C₁-C₁₀ alkyl radical, and/or two or more R* may join together to form a substituted or unsubstituted completely saturated, partially unsaturated, or aromatic cyclic or polycyclic ring structure.

In one or more aspects of the disclosure, the final product may further include one or more flavoring agents, for example, any compound that can add flavor upon dilution into an aqueous liquid. Exemplary flavoring agents include those used to produce fruit flavors, such as guava, kiwi, peach, mango, papaya, pineapple, banana, strawberry, raspberry, blueberry, orange, grapefruit, tangerine, lemon, lime and lemon-lime; cola flavors, tea flavors, coffee flavors, chocolate flavors, dairy flavors, root beer and birch beer flavors, methyl salicylate (wintergreen oil, sweet birch oil), citrus oils and other flavors. Typically, the flavors are safe and/or desirable for human consumption, for example, GRAS or Kosher-certified flavors. Exemplary flavoring agents that can be used in the composition include lemon oil, for example lemon oil sold by Mission Flavors (Foothill Ranch, Calif.), and D-limonene, for example, 99% GRAS certified D-Limonene, sold by Florida Chemical (Winter Haven, Fla.).

In one aspect of the disclosure, the final product may be appropriately characterized as being “generally recognized as safe” for human consumption (“GRAS”).

### Aspects Listing

Accordingly, the instant disclosure provides the following aspects
A1. A process comprising:
   a) contacting a Cannabis sativa plant comprising a first amount of cannabinoids with a solvent including C₁-C₅ hydrocarbons to produce an extract mixture and separating the Cannabis sativa plant from the extract mixture to form a crude extract including less than or equal to about 1 wt % of triglycerides having C₆-C₃₀ fatty acid residues and/or C₆-C₃₀ fatty acids, based on the total amount of the Cannabis sativa plant present;
   b) removing the solvent from the crude extract to produce a first extract; c) combining the first extract with an ethanolic solvent to produce a first extract mixture;
   d) contacting the first extract mixture with a cyclodextrin to produce a second mixture including a cyclodextrin-cannabinoid complex;
   e) combining the second mixture with an aqueous solution including an amino acid selected from the group consisting of glycine, alanine, serine, or a combination thereof, to produce a final product mixture comprising an amino acid-cyclodextrin-cannabinoid complex; and
   f) removing the solvent from the final product mixture to produce a final solid product including the amino acid-cyclodextrin-cannabinoid complex, having greater than or equal to about 90 wt % of the cannabinoids present in the first amount of cannabinoids.
A2. The process of aspect A1, wherein the crude extract is produced by repeating step (a) a plurality of times using the same Cannabis sativa plant and fresh solvent, each time producing a partial crude extract, and combining the plurality of partial crude extracts to form the crude extract.
A3. The process of aspect A1 or A2, wherein the one or more Cannabis sativa plants are contacted with the solvent at a temperature of less than or equal to about 40° C. for a period of time less than or equal to about 10 minutes.
A4. The process of any one of aspects A1 through A3, wherein step (b) includes heating the crude extract at temperature of less than or equal to about 60° C. at less than or equal to atmospheric pressure.
A5. The process of any one of aspects A1 through A4, wherein the first extract mixture consists essentially of the first extract and absolute ethanol and is a clear solution at 25° C.
A6. The process of any one of aspects A1 through A5, wherein step d) includes combining the first extract mixture with cyclodextrin dissolved in absolute ethanol under homogenizing conditions at a temperature below about 30° C. to form the second mixture, and wherein the second mixture is a clear solution at 25° C.
A7. The process of any one of aspects A1 through A6, wherein step (e) includes combining the second mixture with the aqueous amino acid solution including less than or equal to about 10 wt % glycine under homogenizing conditions at a temperature below about 30° C. and the final product mixture is an emulsion, and wherein a 10 wt % mixture of the final solid product in water forms a clear solution at 25° C.
A8. The process of any one of aspects A1 through A7, wherein the amino acid-cyclodextrin-cannabinoid complex includes a cyclodextrin-cannabinoid/terpene/flavonoid complex at least partially encapsulated with the amino acid.
A9. The process of any one of aspects A1 through A8, wherein the solvent is removed from the final product mixture by thin film evaporation at less than or equal to about 30° C. and less than atmospheric pressure, by lyophilization, or a combination thereof.
A10. The process of any one of aspects A1 through A9, further comprising mastication of the final solid product into a free-flowing powder.
A11. The process of any one of aspects A1 through A10, wherein a 10 wt % mixture of the final solid product in water forms a clear solution at 25° C.
A12. The process of any one of aspects A1 through A11, further comprising capturing at least a portion of the solvent removed during one or more steps, optionally purifying the solvent, and then directing at least a portion of the solvent back into the process.
A13. The process of any one of aspects A1 through A12, wherein the cyclodextrin comprises one or more cyclodextrins substituted with one or more functional groups selected from the group consisting of C₁-C₂₀ saturated hydrocarbon radicals, C₃-C₂₀ unsaturated hydrocarbon radicals, C₆-C₂₀ aromatic hydrocarbon radicals, C₅-C₂₀ heterocyclic hydrocarbon radicals, Br, Cl, F, I, —NR*₂, —NR*—CO—R*,—OR*,*—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, where q is 1 to 10 and each R* is independently hydrogen, a C₁-C₂₀ radical, a substituted C₁-C₂₀ radical, and/or two or more R* may join together to form a substituted or unsubstituted completely saturated, partially unsaturated, aromatic, cyclic, or polycyclic ring structure.
A14. The process of any one of aspects A1 through A13, wherein the cyclodextrin is selected from the group consisting of randomly methylated beta-cyclodextrin, randomly methylated gamma-cyclodextrin, 2-hydroxypropyl-beta-cyclodextrin, 2,3-dihydroxypropyl-beta-cyclodextrin, 2-hydroxypropyl-gamma-cyclodextrin, 2,3-dihydroxypropyl-gamma-cyclodextrin, sulfobutyl ether-beta-cyclodextrin, sulfobutyl ether-gamma-cyclodextrin, polymeric cyclodextrin, or a combination thereof.
A15. The process of any one of aspects A1 through A14, wherein the Cannabis sativa plant further includes a first amount of terpenes and/or flavonoids, and wherein the final solid product includes greater than or equal to about 90 wt % of first amount of terpenes and/or flavonoids.
A16. The process of aspect A15, wherein the terpenes and/or flavonoids include one or more of yangonin, epigallocatechin gallate, dodeca-2E,4E,8Z,10Z-tetraenoic acid isobutylamide, dodeca-2E,4E-dienoic acid isobutylamide, beta-myrcene, alpha-pinene, cis-ocimene, beta-curcumene, terpineol, camphene, trans-20 ocimene, alpha-terpinene, piperitone, eucalyptol, linalool, citronellol, beta-pinene, borneol, citronellal, geraniol, d/l-fenchone, d-limonene, 3-carene, geranyl acetate, cuminaldehyde, alpha-phellandrene, alpha-thujone, d/l-menthol, linalyl acetate, isopulegol, carvone, cavacrol, gamma-terpinene, nerol, menthofuran, sabinene hydrate, sabinene, thymol, camphor, pulegone, bornyl acetate, alpha-terpinol, meta-cymene, cannabispiran, isocannabispiran, beta-farnesene, isoborneol, cis-citral, beta-caryophylene, beta-caryophylene oxide, ledene, alpha-humlene, beta-cedrene, alpha-bisabolol, valencene, cedral, farnesol, cuparene, gauiol, thujopene, phloroglucinol, cannabistillbene, 2-carene, quercetin, cannflavin C, isocannabispiran, luteolin, vitexin, cannflavin A, cannflavin B, cytisoide, apigenin, apigenin-glucoside, isovitexin, dihydo-resveratrol, kaempferol, terpinolene, and vincenin-2.
A17. The process of aspect A15 or A16, wherein the terpenes and/or flavonoids include one or more of d-limonene, beta-pinene, alpha-pinene, beta-myrcene, linalool, beta-caryophylene, alpha-humlene, citronellol, terpinolene, borneol, carvone, 2-piperidone, nerolidol, carene, ocimene, cymene, eucalyptol, and pulegone.
A18. The process of any one of aspects A1 through A17, wherein the Cannabis sativa plant extracted in step a) has been maintained at a temperature of less than or equal to about 10° C. from a time of less than 24 hrs after harvest to the time of the extraction.
A19. A composition comprising the final solid product produced by the process of any one of aspects A1 through A18.

### EXAMPLES

The following examples further demonstrate an aspect of the disclosure, and are presented for demonstration and are not to be considered as limiting the disclosure.

An amount of flash frozen Cannabis sativa plants were obtained from a harvester. The plant material was loaded into a stainless steel extraction vessel and extracted 3 times for less than 20 minutes each using propane at about 10° C. The extract mixture was filtered to remove the plant material and each of the crude extracts combined to form a single crude extract.

The solvent was removed from the crude extract via simple distillation to produce an amber oil. This oil was then purged with compressed air at about 15.5° C. for a period of about 1 hour and then placed in a vacuum oven at about 50° C. and 28.5 inches of Hg vacuum for about 3 days to produce about 50 g of the first extract.

This extract with then dissolved in about 250 mls of absolute ethanol to form a clear solution (the first extract mixture) with stirring. This first extract mixture was then combined with a solution of about 260 g of hydroxy-propyl beta cyclodextrin (HPBCD) (mw 1396/g/mol). The amount of HPBCD utilized was determined utilizing an average molecular weight of the first extract as follows.

An average of various analysis of first extracts resulted in the results presented in Table 1.

| | | MASS % | MASS % |
|---|---|---|---|
| | | BEFORE | AFTER |
| | MOL. | DECARBOX- | DECARBOX- |
| CANNABINOID | WEIGHT | YLATION | YLATION |
| THCa | 358.48 | 86.84 | |
| Δ⁹-THC | 314.469 | 3.92 | 80.08 |
| Δ⁸-THC | 314.469 | <0.01 | |
| THCVa | 286.4 | NR | |
| THCV | 242.4 | NR | |
| CBDa | 358.48 | 0.44 | |
| CBD | 314.469 | <0.01 | 0.39 |
| CBDVa | 330.4 | <0.01 | |
| CBDV | 286.4 | NR | |
| CBN | 310.44 | <0.01 | |
| CBGa | 360.49 | 0.79 | |
| CBG | 316.49 | NR | 0.69 |
| CBCa | 358.47 | NR | |
| CBC | 314.47 | NR | |
| CBLa | 330.42 | NR | |
| SUMMATION | | 91.99 | 81.16 |

The mass % after decarboxylation (the fourth column) was calculated from these data to correct for the decarboxylation that is known to occur as a result of the process. Accordingly, the net THC content is found through the formula: THC=(0.877)(THCa)+THC. 2) The decarboxylation allows for loss of CO₂ but not of any Terpenes or Cannabinoids. Accordingly, the mass % of Terpenes after decarboxylation would be 100.00%-81.16%=18.84%. The average analysis further include quantification of various terpenes in which the mass percent are shown as mass % of the overall terpene profile and not of the total mass of oil. The mass percentage is The following chart was included. The last column was utilized to determine the actual mass percent of the terpenes and the contribution made to the average molecular weight of the extract.

| | | | Mass % of Oil |
|---|---|---|---|
| | MOL. | Mass % of | based on 18.84% |
| TERPENE | WT. | Terpenes | Terpene Content |
| Myrcene | 136.23 | 54.21 | (18.84)(0.5421) = 10.2 |
| α-Pinene | 136.23 | 31.69 | (18.84)(0.3169) = 5.97 |
| Limonene | 136.23 | 6.73 | (18.84)(0.0673) = 1.27 |
| β-Pinene | 136.23 | 6.15 | (18.84)(0.0615) = 1.16 |
| Carene | 136.23 | 0.34 | (18.84)(0.0034) = 0.064 |
| α-Terpinene | 136.23 | 0.33 | (18.84)(0.0033) = 0.062 |
| p-Cymene | 134.222 | 0.22 | (18.84)(0.0022) = 0.041 |
| β-Caryophyllene | 204.36 | 0.16 | (18.84)(0.0016) = 0.030 |
| Humulene | 204.35 | 0.12 | (18.84)(0.0012) = 0.023 |
| Nerolidol | 222.37 | 0.04 | (18.84)(0.0004) = 0.008 |

Assuming the difference between the contribution of terpenes and the actual contribution of the terpenes, flavonoids, and other materials present is negligible, the average molecular weight of the oil was calculated as a weighted average of each compound using their molecular weights and their mass percentages. Based on these data, the average molecular weight of the first extract is equal to the sum of the mass percentage of the component multiplied by the molecular weight of that component normalized to 100% as follows:

(0.8008)(314.469)+(0.0039)(314.469)+(0.0069)(316.49)+(0.102)(136.23)+(0.0597)(136.23)+(0.0127)(136.23)+(0.0116)(136.23)+(0.00064)(136.23)+(0.00062)(136.23)+(0.00041)(134.222)+(0.00030)(204.36)+(0.00023)(204.35)+(0.00008)(222.37)=280.928 g/mol.

Note: the assumption that the flavonoids represent a negligible fraction of the overall composition appears valid due to the lack of oiling out of the material after forming the cannabinoid-cyclodextrin product—which was a clear homogeneous solution.

Accordingly, the amount of HPBCD required was calculated as follows:

1) Moles of oil molecules:

$$50.00 g oil \times \frac{1 mol oil molecules}{280.928 g} = 0.17798 mol oil molecules$$

2) Using 1.025 molar excess of HPBCD:

$$( 1.05 e q ) ( 0.17798 mol ) = 0.187 mol HPBCD$$

$$0.187 mol HPBCD \times \frac{1396 g HPBCD}{1 mol HPBCD} = 260.0 g HPBCD$$

3) For this oil @ 80.08% THC (50.00 g oil)(0.8008)=40.04 g THC=40,040 mg THC
4) To ensure at least 100 mg THC/g final product a 6% excess was calculated:

$$\frac{40.040 mg THC}{106 mg THC / g Final product} = 377 g Final Product$$

5) Mass glycine: 377 g Final product-260 g HPBCD-50.00 g oil=67 g glycine
6) Volume of solvents: ˜15 mls EtOH(abs) per 1 g HPBCD=3,900 mL ETOH(abs)
˜4.5 mls EtOH(abs) per 1 g oil=225 mL ETOH(abs)
˜2 mls H₂O per 1 g glycine=134 mL H₂O

The above step of determining the amount of complexing agent to utilize provides a significant benefit over simply utilizing the molecular weight of THC, 314.469 g/mol, which would have resulted in an insufficient number of host molecules and uncomplexed oil compounds which would result in waist. This further allows for more precise utilization of the expensive HPBCD.

The first extract mixture was combined with the HPBCD solution and homogenized at about 18,000 RPM for about 3 hours at a temperature from about 10° C. to less than 22° C. to form a clear solution. A 5 wt % aqueous solution of glycine was then added and the mixture homogenized again for 3 hours at a temperature from about 10° C. to about 20° C. The resulting solution was lyophilized overnight at about 10° C. to 15° C. to form an off-white solid. The solid was ground into a fine power. A 10 wt % aqueous solution of the powered in water at 25° C. produced a clear colorless solution. Further analysis of the material indicated ˜98 wt % of the cannabinoids originally present in the plant material were present in the final product, along with over 95 wt % of the terpenes and other desirable components.

The above description is made for the purpose of illustrating the general principles of the present disclosure and is not meant to limit the inventive concepts claimed herein. Further, particular features described herein can be used in combination with other described features in each of the various possible combinations and permutations.

It should be noted that in the development of any such actual aspect, numerous implementation-specific decisions must be made to achieve the developer's specific goals, such as compliance with system related and business related constraints, which will vary from one implementation to another. Moreover, it will be appreciated that such a development effort might be complex and time consuming but would nevertheless be a routine undertaking for those of ordinary skill in the art having the benefit of this disclosure.

In addition, the device, system and/or method used/disclosed herein can also comprise some components other than those cited.

Unless otherwise specifically defined herein, all terms are to be given their broadest possible interpretation including meanings implied from the specification as well as meanings understood by those skilled in the art and/or as defined in dictionaries, treatises, and the like.

It must also be noted that, as used in the specification and the appended claims, the singular forms “a,” “an” and “the” include plural referents unless otherwise specified.

As also used herein, the term “about” denotes an interval of accuracy that ensures the technical effect of the feature in question. In various approaches, the term “about” when combined with a value, refers to plus and minus 10% of the reference value. For example, a thickness of about 10 angstroms (Å) refers to a thickness of 10 Å+/−1 Å, e.g., from 0.9 Å to 1.1 Å in this example.

In the summary and this detailed description, each numerical value should be read once as modified by the term “about” (unless already expressly so modified), and then read again as not so modified unless otherwise indicated in context. Also, in the summary and this detailed description, it should be understood that a physical range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific data points within the range, or even no data points within the range, are explicitly identified or refer to only a few specific, it is to be understood that inventors appreciate and understand that any and all data points within the range are to be considered to have been specified, and that inventors possessed knowledge of the entire range and all points within the range.

As used in the specification and claims, “near” is inclusive of “at.” “The term “and/or” refers to both the inclusive “and” case and the exclusive “or” case, and such term is used herein for brevity. For example, a composition comprising “A and/or B” may comprise A alone, B alone, or both A and B.

Various components described in this specification may be described as “including” or made of certain materials or compositions of materials. In one aspect, this can mean that the component consists of the particular material(s). In another aspect, this can mean that the component comprises the particular material(s).

The word “exemplary” is used herein to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation.

While various aspects have been described above, it should be understood that they have been presented by way of example only, and not limitation. Thus, the breadth and scope of an aspect of the present invention should not be limited by any of the above-described exemplary aspects, but should be defined only in accordance with the following claims and their equivalents.

We claim:

1. A process comprising:
a) contacting a Cannabis sativa plant comprising a first amount of cannabinoids with a solvent including C₁-C₅ hydrocarbons to produce an extract mixture and separating the Cannabis sativa plant from the extract mixture to form a crude extract including less than or equal to about 1 wt % of triglycerides having C₆-C₃₀ fatty acid residues and/or C₆-C₃₀ fatty acids, based on the total amount of the Cannabis sativa plant present;
b) removing the solvent from the crude extract to produce a first extract;
c) combining the first extract with an ethanolic solvent to produce a first extract mixture;
d) contacting the first extract mixture with a cyclodextrin to produce a second mixture including a cyclodextrin-cannabinoid complex;
e) combining the second mixture with an aqueous solution including an amino acid selected from the group consisting of glycine, alanine, serine, or a combination thereof, to produce a final product mixture comprising an amino acid-cyclodextrin-cannabinoid complex; and
f) removing the solvent from the final product mixture to produce a final solid product including the amino acid-cyclodextrin-cannabinoid complex, having greater than or equal to about 90 wt % of the cannabinoids present in the first amount of cannabinoids.

2. The process of claim 1, wherein the crude extract is produced by repeating step (a) a plurality of times using the same Cannabis sativa plant and fresh solvent, each time producing a partial crude extract, and combining the plurality of partial crude extracts to form the crude extract.

3. The process of claim 1, wherein the one or more Cannabis sativa plants are contacted with the solvent at a temperature of less than or equal to about 40° C. for a period of time less than or equal to about 10 minutes.

4. The process of claim 3, wherein step (b) includes heating the crude extract at temperature of less than or equal to about 60° C. at less than or equal to atmospheric pressure.

5. The process of claim 4, wherein the first extract mixture consists essentially of the first extract and absolute ethanol and is a clear solution at 25° C.

6. The process of claim 5, wherein step d) includes combining the first extract mixture with cyclodextrin dissolved in absolute ethanol under homogenizing conditions at a temperature below about 30° C. to form the second mixture, and wherein the second mixture is a clear solution at 25° C.

7. The process of claim 6, wherein step (e) includes combining the second mixture with the aqueous amino acid solution including less than or equal to about 10 wt % glycine under homogenizing conditions at a temperature below about 30° C. and the final product mixture is an emulsion, and wherein a 10 wt % mixture of the final solid product in water forms a clear solution at 25° C.

8. The process of claim 7, wherein the amino acid-cyclodextrin-cannabinoid complex includes a cyclodextrin-cannabinoid/terpene/flavonoid complex at least partially encapsulated with the amino acid.

9. The process of claim 7, wherein the solvent is removed from the final product mixture by thin film evaporation at less than or equal to about 30° C. and less than atmospheric pressure, by lyophilization, or a combination thereof.

10. The process of claim 1 further comprising mastication of the final solid product into a free-flowing powder.

11. The process of claim 1, wherein a 10 wt % mixture of the final solid product in water forms a clear solution at 25° C.

12. The process of claim 1, further comprising capturing at least a portion of the solvent removed during one or more steps, optionally purifying the solvent, and then directing at least a portion of the solvent back into the process.

13. The process of claim 1, wherein the cyclodextrin comprises one or more cyclodextrins substituted with one or more functional groups selected from the group consisting of C₁-C₂₀ saturated hydrocarbon radicals, C₃-C₂₀ unsaturated hydrocarbon radicals, C₆-C₂₀ aromatic hydrocarbon radicals, C₅-C₂₀ heterocyclic hydrocarbon radicals, Br, Cl, F, I, —NR*₂, —NR*—CO—R*,—OR*,*—O—CO—R*, —CO—O—R*, —SeR*, —TeR*, —PR*₂, —PO—(OR*)₂, —O—PO—(OR*)₂, —AsR*₂, —SbR*₂, —SR*, —SO₂—(OR*)₂, —BR*₂, —SiR*₃, —GeR*₃, —SnR*₃, —PbR*₃, —(CH₂)q-SiR*₃, or a combination thereof, where q is 1 to 10 and each R* is independently hydrogen, a C₁-C₂₀ radical, a substituted C₁-C₂₀ radical, and/or two or more R* may join together to form a substituted or unsubstituted completely saturated, partially unsaturated, aromatic, cyclic, or polycyclic ring structure.

14. The process of claim 1, wherein the cyclodextrin is selected from the group consisting of randomly methylated beta-cyclodextrin, randomly methylated gamma-cyclodextrin, 2-hydroxypropyl-beta-cyclodextrin, 2,3-dihydroxypropyl-beta-cyclodextrin, 2-hydroxypropyl-gamma-cyclodextrin, 2,3-dihydroxypropyl-gamma-cyclodextrin, sulfobutyl ether-beta-cyclodextrin, sulfobutyl ether-gamma-cyclodextrin, polymeric cyclodextrin, or a combination thereof.

15. The process of claim 1, wherein the Cannabis sativa plant further includes a first amount of terpenes and/or flavonoids, and wherein the final solid product includes greater than or equal to about 90 wt % of first amount of terpenes and/or flavonoids.

16. The process of claim 15, wherein the terpenes and/or flavonoids include one or more of yangonin, epigallocatechin gallate, dodeca-2E,4E,8Z,10Z-tetraenoic acid isobutylamide, dodeca-2E,4E-dienoic acid isobutylamide, beta-myrcene, alpha-pinene, cis-ocimene, beta-curcumene, terpineol, camphene, trans-20 ocimene, alpha-terpinene, piperitone, eucalyptol, linalool, citronellol, beta-pinene, borneol, citronellal, geraniol, d/l-fenchone, d-limonene, 3-carene, geranyl acetate, cuminaldehyde, alpha-phellandrene, alpha-thujone, d/l-menthol, linalyl acetate, isopulegol, carvone, cavacrol, gamma-terpinene, nerol, menthofuran, sabinene hydrate, sabinene, thymol, camphor, pulegone, bornyl acetate, alpha-terpinol, meta-cymene, cannabispiran, isocannabispiran, beta-farnesene, isoborneol, cis-citral, beta-caryophylene, beta-caryophylene oxide, ledene, alpha-humlene, beta-cedrene, alpha-bisabolol, valencene, cedral, farnesol, cuparene, gauiol, thujopene, phloroglucinol, cannabistillbene, 2-carene, quercetin, cannflavin C, isocannabispiran, luteolin, vitexin, cannflavin A, cannflavin B, cytisoide, apigenin, apigenin-glucoside, isovitexin, dihydo-resveratrol, kaempferol, terpinolene, and vincenin-2.

17. The process of claim 15 wherein the terpenes and/or flavonoids include one or more of d-limonene, beta-pinene, alpha-pinene, beta-myrcene, linalool, beta-caryophylene, alpha-humlene, citronellol, terpinolene, borneol, carvone, 2-piperidone, nerolidol, carene, ocimene, cymene, eucalyptol, and pulegone.

18. The process of claim 1, wherein the Cannabis sativa plant extracted in step a) has been maintained at a temperature of less than or equal to about 10° C. from a time of less than 24 hrs after harvest to the time of the extraction.

19. A composition comprising the final solid product produced by the process of claim 1.

20. A composition comprising the final solid product produced by the process of claim 7.
